(19) United States
(12) Patent Application Publication
(10) Pub. No.: US 2004/0266794 A1
(43) Pub. Date: Dec. 30, 2004

(54) Pramipexole for the reduction of excessive food intake for children

(75) Inventors: Joachim Mierau, Mainz (DE); Juergen Reess, Ulm (DE); Marion Wienrich, Weiterstadt (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/801,286

(22) Filed: Mar. 16, 2004

Related U.S. Application Data
(60) Provisional application No. 60/496,747, filed on Aug. 21, 2003.

(30) Foreign Application Priority Data
Mar. 21, 2003 (DE) DE 103 12 809

(51) Int. Cl. A61K 31/52

(52) U.S. Cl. 514/263.1; 514/419

(57) ABSTRACT

The invention relates to the use of dopamine receptor agonists for preparing a pharmaceutical composition for reducing excessive food intake.

### RELATED APPLICATIONS

Benefit of U.S. Provisional Application Ser. No. 60/496,747, filed on Aug. 21, 2003 is hereby claimed, and which application is incorporated herein in its entirety.

### FIELD OF INVENTION

The invention relates to the use of pramipexol for producing a medicament for reducing excessive food intake in children.

### BACKGROUND TO THE INVENTION

Excessive food intake generally leads to overweight or obesity, i.e. an increase in normal weight exceeding normal limits. Being overweight currently constitutes not only an excessively high health risk but also a social problem, particularly in children. Being overweight is a risk factor for a number of subsequent diseases such as high blood pressure, diabetes mellitus, hyperlipidaemia, arthritis, gout and the attendant vascular diseases, particularly arteriosclerosis. In addition, even in children, being overweight can lead to emotional problems even extending to depression.

Overweight and obesity can be diagnosed in children using gender-specific age percentiles for the BMI (body mass index) (Leitlinien der DGFKJ, Urban and Fischer, January 2002).

The only effective therapeutic measure is a reduction in the calorie intake. This is difficult to achieve in many young patients in spite of a knowledge of the consequences mentioned above.

The aim of the invention is to make it easier for young patients ranging from 6 to 18 years old to reduce their calorie intake and thus reduce the health risks associated with being fat.

### DESCRIPTION OF THE INVENTION

It has surprisingly been shown that pramipexol can be used effectively for reducing excessive food intake in children in doses which are therapeutically well tolerated.

Accordingly, the present invention relates to the use of pramipexol for producing a medicament for reducing excessive food intake in children.

Preferably, pramipexol is used to produce a medicament for reducing excessive food intake in children ranging from 6 to 18 years old, preferably from 12 to 18 years old.

It is also preferably used in children whose BMI is above the 90th percentile, preferably above the 97th percentile.

It is particularly preferable to use pramipexol by administering it to the children in a daily dose of about 0.005 mg/kg to 0.02 mg/kg of body weight, preferably about 0.1 mg/kg of body weight.

It is also particularly preferable to use pramipexol to produce a medicament for treating obesity in Type 2 diabetes in children.

It is particularly preferable to use pramipexol to produce a medicament for continuous administration to children.

It is also particularly preferable to use pramipexol to produce a medicament for transdermal administration to children.

It is also preferable to use pramipexol optionally in the form of its enantiomers, preferably the (−)-enantiomer, optionally in the form of the pharmacologically acceptable acid addition salts and optionally in the form of the hydrates and solvates thereof.

It is particularly important to use a pharmaceutical composition containing as active substance pramipexol optionally in the form of the enantiomers thereof, preferably the (−)-enantiomer, optionally in the form of the pharmacologically acceptable acid addition salts and optionally in the form of the hydrates and solvates or the physiologically acceptable salts thereof combined with one or more active substances selected from among the dopamine-D₁, D₂, D₃ or D₄ agonists, anorectics, lipase inhibitors and sympathomimetics for preparing a pharmaceutical composition for treating children.

Pramipexol has a high selectivity for the dopamine-D₃ receptor. It can be shown that this reduces the side effects of a pharmacological influence on food intake. The D₃ receptor is located predominantly in those regions of the brain which are associated with emotion. Activation of the D₃ receptor by pramipexol can contribute to a reduction in excessive food intake or pathologically disrupted food intake by lightening the mood.

The pramipexol used within the scope of the present invention may optionally be used in the form of its enantiomers or racemates, optionally in the form of the pharmacologically acceptable acid addition salts and optionally in the form of the hydrates and solvates.

References to pramipexol include the (+)-enantiomer as well as the racemate. Within the scope of the present invention the (−)-enantiomer is of particular significance.

The pramipexol which may be used according to the invention may optionally be used in the form of its pharmaceutically acceptable acid addition salts and optionally in the form of the hydrates and/or solvates. By pharmaceutically acceptable acid addition salts of pramipexol are meant, according to the invention, those salts which are selected from the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid, while the salts of hydrochloric acid, hydrobromic acid, sulphuric acid, phosphoric acid and acetic acid are particularly preferred. The salts of hydrochloric acid are of particular significance.

In this respect pramipexol dihydrochloride is of particular importance. For transdermal application the pramipexol base is preferably used. Of the hydrates of pramipexol, pramipexol dihydrochloride monohydrate is particularly preferred.

According to the invention, pramipexol may optionally be used in conjunction with other active substances. Preferred combination partners are compounds selected from the categories of the dopamine-D₁, D₂, D₃ or D₄ receptor agonists, selected from the group consisting of Adrogolide, A-86929, Rotigotine, NeurVex, Nolomirole, Talipexole, CHF 1512, (−)-Stepholidine, DAR-201, Diacrin/Genzyme, Bromocriptine, Bupropion, LEK-8829, BAM-1110, AIT-203, NS-2330, Terguride, Aripiprazole, OPC-4392, GMC-1111, PD-148903, Apomorphine HCI, PD-89211, PD-158771, Cabergoline, Sumanirole, PNU-14277E, POL-255, Dihydrexidine, GBR-12783, Quinagolide HCI, (R)-Bupropion, S-32504, S-33592, SKF-80723, SKF-83959, Fenoldopam, Ropinirole, SKF-82958, SKF-77434, DU 127090, SLV-308, SLV 318, NeuroCRIB, SP-1037C, Spheramine, Gallotrank, Preclamol, DAB-452, YM-435, BP-897, ProSavin, Etilevodopa, P63, A 68930, A 77636, Alaptide, Alentemol, CI 1007; PD 143188, BLSI, JA 116a; JA 116, Melevodopa; Levodopa methyl; CHF 1301; NSC 295453; Levomet, MR 708, PD 128483, RD 211, SKF 38393, SKF 81297, U 86170F, U 91356A, WAY 124486 and Z 15040, the antidepressants, the anorectics, preferably silbutramine, the lipase inhibitors, preferably orlistat, and the sympathomimetics, preferably ephedrine. Thanks to the synergistic effects in the intended activity, the dosage of the individual components can be reduced, when using combinations containing the dopamine-receptor agonists according to the invention together with one of the other active substances mentioned above.

The activity according to the invention will now be illustrated using the following example of pramipexol. It is intended merely to illustrate the invention and must not be regarded as limiting it.

Effect of Pramipexol on Food Intake in Mice

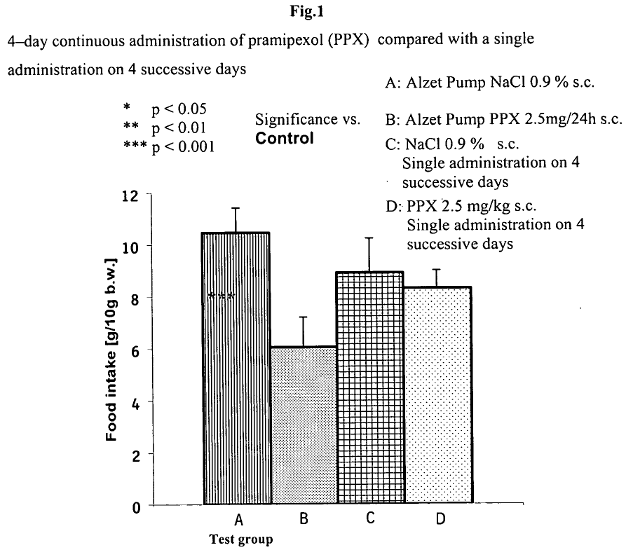

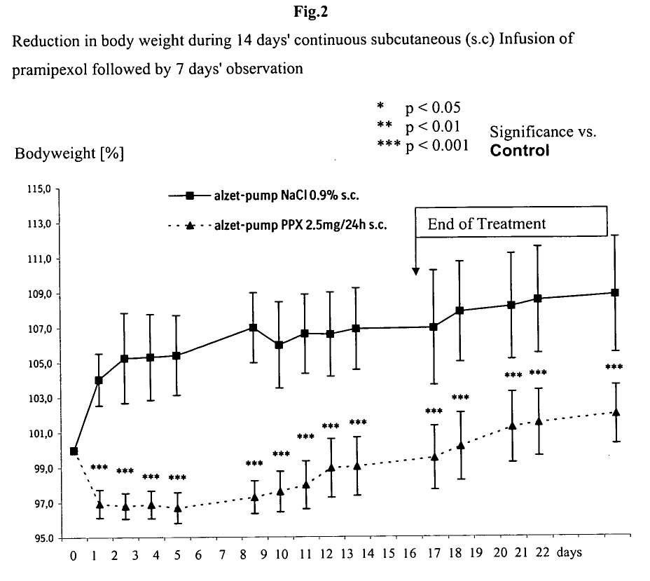

Pramipexol inhibits food intake in mice when administered continuously. Continuous administration using osmotic pumps led to a permanent, statistically highly significant inhibition in food intake (FIG. 1). By contrast, a single application given on successive days, at a dosage comparable to that given by long term administration, did not significantly reduce food intake (FIG. 1). Moreover, in long term administration, a permanent weight reduction was observed which was statistically highly significant even after the pramipexol treatment had ended (FIG. 2).

Test method by single administration:

10 mice (Strain: C57BL/6) were deprived of food for 24 hours while being given free access to drinking water.

20 minutes before the end of the fasting period pramipexol was administered (2.5 mg/kg of body weight s.c.). The control group, also 10 mice, were given physiological saline solution, the solvent used for pramipexol. Then the animals were offered food and the food consumption over 4 days in 30 minute cycles was measured.

Test method for continuous administration:

10 mice (Strain: C57BL/6) were deprived of food for 24 hours while being given free access to drinking water.

20 minutes before the end of the fasting period an alzet® Mini-osmotic pump (Model 2002) was implanted subcutaneously in the animals, with a dosage release of 2.5 mg of pramipexol/24 hours s.c. The pumping rate was 0.54 μl/h. A group of 10 control animals were given the solvent, physiological saline, at the same pumping rate in an analogous experiment. The continuous release of the substance or solvent was measured for 4 days. The food intake was measured over the first 10 hours at 2 hour intervals and later daily.

The change in weight with continuous administration was measured over a period of 22 days, the administration of pramipexol ending after 14 days. The change in weight was measured every day.

Without restricting the subject of the present invention to this, some possible doses for administering pramipexol to children will now be given. The substance may be used in doses of about 0.05 to 3 mg, preferably about 0.1 to 1.5 mg per day. These doses are based on pramipexol in the form of its free base. Based on the salt form pramipexol dihydrochloride monohydrate which is preferably used, the doses mentioned above correspond to about 0.07 to 4.26 mg, preferably 0.14 to 2.13 mg of pramipexol dihydrochloride monohydrate per day.

One possible dosing method, which is described solely by way of example, is given below (based on pramipexol in the form of its free base): Individual dosage titration at weekly intervals depending on potency and compatibility.

1. First Week: 1 tablet containing 0.088 mg of pramipexol 3 times a day;

2. Second Week: 1 tablet containing 0.18 mg of pramipexol 3 times a day;

3. Third Week and thereafter: Half a tablet containing 0.7 mg of pramipexol 3 times a day.

Pramipexol may be administered, for the purposes of the invention, by oral, transdermal, intrathecal route, by inhalation, nasally or parenterally, preferably transdermally or parenterally, most preferably transdermally. Suitable formulations include for example tablets, preferably slow release tablets, capsules, suppositories, solutions, syrups, emulsions, dispersible powders, implants or plasters, preferably plasters, most preferably micronal plasters. Regarding possible embodiments of a transdermal form for use according to the invention, reference is hereby made, with regard to pramipexol, to the embodiments according to U.S. Pat. No. 5,112,842, which are specifically mentioned hereby. Tablets may for example be obtained by mixing the active substance or substances with known excipients, e.g. inert diluents such as calcium carbonate, calcium phosphate or lactose, disintegrants such as corn starch or alginic acid, binders such as starch or gelatine, lubricants such as magnesium stearate or talc, and/or agents for obtaining delayed release such as carboxymethylcellulose, cellulose acetate phthalate, or polyvinyl acetate. The tablets may also be made up of several layers.

The following are some examples of pharmaceutical preparations which may be used according to the invention. These are provided solely as an illustration without restricting the subject matter of the invention thereto.

Tablet 1

| Ingredients: | mg |
|---|---|
| Pramipexol dihydrochloride-monohydrate | 1.0 |
| Mannitol | 121.50 |
| Maize starch | 79.85 |
| Highly dispersed silicon dioxide, anhydrous | 2.30 |
| Polyvidone K25 | 2.35 |
| Magnesium stearate | 3.00 |
| Total | 210.00 |

Tablet 2

| Ingredients: | mg |
|---|---|
| Pramipexol | 0.5 |
| Mannitol | 122.0 |
| Maize starch, dried | 61.8 |
| Maize starch | 18.0 |
| Highly dispersed silicon dioxide, anhydrous | 2.4 |
| Polyvidone K25 | 2.3 |
| Magnesium stearate | 3.0 |
| Total | 210.0 |

Tablet 3

| Ingredients: | mg |
|---|---|
| Pramipexol | 0.25 |
| Mannitol | 61.00 |
| Maize starch | 39.90 |
| Highly dispersed silicon dioxide, anhydrous | 1.20 |
| Polyvidone K25 | 1.15 |
| Magnesium stearate | 1.5 |
| Total | 105.00 |

Tablet 4

| Ingredients: | mg |
|---|---|
| Pramipexol | 0.125 |
| Mannitol | 49.455 |
| Maize starch, dried | 25.010 |
| Maize starch | 7.300 |
| Highly dispersed silicon dioxide, anhydrous | 0.940 |
| Polyvidone K25 | 0.940 |
| Magnesium stearate | 1.230 |
| Total | 85.000 |

Solution for Injection:

| | | |
|---|---|---|
| Pramipexol dihydrochloride monohydrate | 0.3 | mg |
| Sodium chloride | 0.8 | mg |
| Benzalkonium chloride | 0.01 | mg |
| Water for injections ad | 100 | ml |

1) a method for reducing food consumption in a subject ranging from 6 to 18 years old comprising administering to the subject a pharmaceutical composition comprising an effective amount of pramipexole.

2) The method according to claim 1 wherein the subject ranges from 12 to 18 years old.

3) The method according to claim 1 wherein the subject has a BMI above the 90th percentile.

4) The method according to claim 1 wherein the subject is given a daily dose of 0.005 to 0.02 mg of pramipexol per kg of body weight.

5) A method for treating obesity in a subject ranging from 6 to 18 years old and having type 2 diabetes comprising administering to the subject a pharmaceutical composition comprising an effective amount of pramipexole.

6) The method according to claim 1 comprising continuous administration of the composition to the subject.

7) The method according to claim 6 wherein the continuous administration comprises transdermal administration.

8) The method according to claim 1 wherein pramipexol is optionally used in the form of its enantiomer, optionally in the form of the pharmacologically acceptable acid addition salts and optionally in the form of the hydrates and solvates, or the physiologically acceptable salts thereof.

9) The method according to claim 8 wherein the pramipexol optionally in the form of its enantiomers, optionally in the form of the pharmacologically acceptable acid addition salts and optionally in the form of the hydrates and solvates or the physiologically acceptable salts thereof is combined with one or more active substances selected from the group consisting of the dopamine-D₁, D₂, D₃ or D₄ agonists, anorectics, lipase inhibitors and sympathomimetics.

10. The method according to claim 5 comprising continuous administration of the composition to the subject.

11. The method according to claim 10 wherein the continuous administration comprises transdermal administration.
